(19) United States
(12) Patent Application Publication
(10) Pub. No.: US 2020/0237438 A1
(43) Pub. Date: Jul. 30, 2020

(54) SKIN TREATMENT LASER APPARATUS USING COMPLEX IRRADIATIONS WITH DIFFERENT PULSE DURATIONS

(71) Applicant: JEISYS MEDICAL INC., Seoul (KR)

(72) Inventor: Suk Bae SEO, Seoul (KR)

(73) Assignee: JEISYS MEDICAL INC., Seoul (KR)

(21) Appl. No.: 16/848,455

(22) Filed: Apr. 14, 2020

Related U.S. Application Data
(63) Continuation of application No. PCT/KR2018/011510, filed on Sep. 28, 2018.

(30) Foreign Application Priority Data
Oct. 16, 2017 (KR) 10-2017-0134121

(51) Int. Cl. A61B 18/20

(52) U.S. Cl. CPC A61B 18/20; A61B 2018/00452

(57) ABSTRACT

Disclosed is a skin treatment laser apparatus using complex irradiations with different pulse durations. A skin treatment laser apparatus using complex radiations with different pulse durations according to the present invention comprises: a laser generation device for modulating at least one reference laser ray into multiple laser rays having different energy magnitudes and pulse durations, and alternating multiple laser rays so as to repeatedly output the alternated laser rays; and a laser irradiation device for irradiating a skin lesion with the multiple laser rays are alternated to be repeatedly applied by the laser generation device, so that laser ays having different pulse durations can be alternately and repeatedly irradiated onto a skin lesion for a laser irradiation period of time according to a chromophore, thereby improving the efficiency of laser treatment.

### CROSS-REFERENCE TO RELATED APPLICATIONS

The present application is a continuation of International Patent Application No. PCT/KR2018/011510, filed on Sep. 28, 2018 which is based upon and claims the benefit of priority to Korean Patent Application No. 10-2017-0134121 filed on Oct. 16, 2017. The disclosures of the above-listed applications are hereby incorporated by reference herein in their entirety.

### BACKGROUND

Embodiments of the inventive concept described herein relate to a skin treatment laser apparatus using complex irradiations with difference pulse durations for improving the efficiency of laser treatment by alternatively and repeatedly radiating laser beams of different pulse durations during a laser irradiation period according to a chromophore.

A laser beam has characteristics capable of being directly radiated without being dispersed and providing a stronger output within a short time at a single wavelength than general beams. Such a laser beam is a non-ionizing beam capable of providing a high output and has characteristics which has excellent monochromaticity and is not dispersed. Thus, absorbing the laser beam into skin tissues or the like causes thermogenic action and photochemical changes to show a phenomenon where a corresponding material is deformed.

Earlier studies of the laser beam in the medical field are conducted on skins and eyes, which are relatively easier to approach than other portions of the human body. As a result, many innovative treatment technologies suitable for treating skin ailments have been developed, and laser treatments have been widely used in dermatological treatments such as pigmented lesions, tattoos, blood vessel lesions, wrinkles, pimples, and skin tightness.

Dermatological laser treatments are based on selective targeting of chromophores in the skin by the normally proper selection of wavelengths and pulse durations of the laser beam.

Different skin defects may include different chromophores. Thus, skin treatment laser devices should be configured to have a plurality of laser sources which provide laser beams for every various wavelength ranges, respectively.

As described above, the laser beam is disclosed to usually provide better treatment results than wideband light sources. However, because laser devices are configured to usually radiate only a laser beam having only a single pulse duration of a single wavelength, when using different wavelength ranges or different durations, a plurality of different laser generators or laser sources, each of which has a different pulse duration for each wavelength range, should be provided.

However, because conventional laser treatment devices, each of which includes a plurality of different laser generators or laser sources for each wavelength range or having different durations, should be complicated in structure, management should be complicated for each laser device and the cost of purchasing the laser treatment device should be greatly increased.

Due to this, because a skin treatment laser beam is repeatedly applied while keeping a laser irradiation period constant, a laser beam used in skin care or the like should be limited to expect an effect over a certain effect because the same impulse is repeatedly provided to a corresponding material.

### SUMMARY

Embodiments of the inventive concept provide a skin treatment laser apparatus using complex irradiations with difference pulse durations for shortening a laser treatment time and improving the efficiency of treatment by alternatively and repeatedly radiating laser beams of different pulse durations during a laser irradiation period according to a chromophore.

According to an exemplary embodiment, a skin treatment laser apparatus capable of performing pulse duration modulation may include a laser generation device that modulates at least one reference laser beam into a plurality of laser beams, each of which has a different energy magnitude and a different pulse duration, and alternately and repeatedly outputs the plurality of laser beams; and a laser irradiation device that irradiates a skin lesion with the plurality of laser beams alternately and repeatedly applied from the laser generation device.

The laser generation device may modulate the at least one reference laser beam into a first laser beam having a first energy magnitude and a first pulse duration and an nth laser beam having an nth energy magnitude and an nth pulse duration different from those of the first laser beam and may alternately and repeatedly output the first to nth laser beams at intervals of a predetermined period.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a configuration diagram illustrating a skin treatment laser apparatus using complex irradiations with difference pulse durations according to an embodiment of the inventive concept;

FIG. 2 is a configuration block diagram illustrating in detail a laser generation device shown in FIG. 1;

FIG. 3 is another configuration block diagram illustrating in detail a laser generation device shown in FIG. 1;

FIG. 4 is another configuration block diagram illustrating in detail a laser generation device shown in FIG. 1;

FIGS. 5A, 5B, and 5C are drawings illustrating changes in pulse duration of a laser beam applied from a laser generation device shown in FIGS. 1 and 2 to a laser irradiation device;

FIGS. 6A, 6B, 6C, and 6D are drawings illustrating changes in pulse duration of a laser beam applied from a laser generation device shown in FIGS. 1 and 3 to a laser irradiation device; and

FIGS. 7A and 7B are drawings illustrating changes in pulse duration of a laser beam applied from a laser generation device shown in FIGS. 1 and 4 to a laser irradiation device.

### DETAILED DESCRIPTION

The foregoing purposes, features, and advantages will be more apparent based on the following detailed description with reference to the accompanying drawings, and therefore a person with ordinary skill in the art to which the inventive concept pertains may easily practice the technical idea of the inventive concept. In describing the inventive concept, when a detailed description of well-known technology relating to the inventive concept may unnecessarily make unclear the spirit of the inventive concept, a detailed description thereof will be omitted. Hereinafter, exemplary embodiments of the inventive concept will be described in detail with reference to the accompanying drawings.

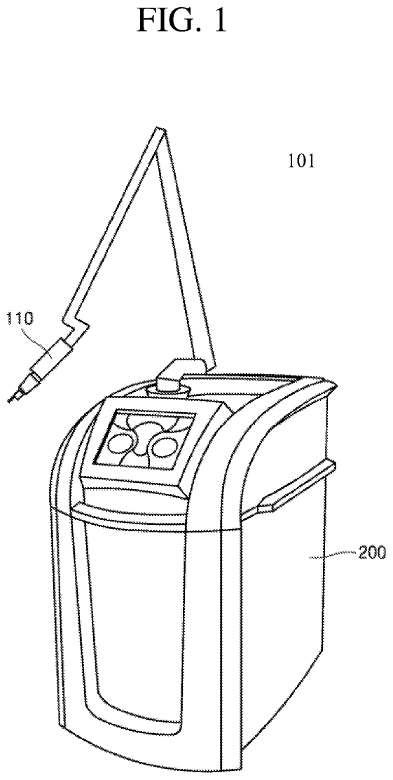

FIG. 1 is a configuration diagram illustrating a skin treatment laser apparatus using complex irradiations with difference pulse durations according to an embodiment of the inventive concept.

A skin treatment laser apparatus 101 shown in FIG. 1 may include a laser generation device 200 for modulating at least one reference laser beam into a plurality of laser beams, each of which has a different energy magnitude and a different pulse duration, and alternately and repeatedly outputting the plurality of laser beams and a laser irradiation device 110 for irradiating a skin lesion with the plurality of laser beams alternatively and repeatedly applied from the laser generation device 200.

The laser generation device 200 may generate a wavelength range of the reference laser beam as a wavelength range such as nanosecond, depending on at least one medium applied to a resonator or the like. The laser generation device 200 may modulate the pulse duration of the reference laser beam such that an energy magnitude of the laser beam is varied according to the pulse duration. The wavelength range of the reference laser beam may be set to a wavelength range, such as picosecond or femtosecond, according to a medium, but, hereinafter, an embodiment of the inventive concept is exemplified as the wavelength range of the reference laser beam is set to the wavelength range of nanosecond for convenience of description.

The laser generation device 200 in an embodiment of the inventive concept may modulate at least one reference laser beam into a plurality of laser beams, each of which has a different energy magnitude and a different pulse duration, and may alternately and repeatedly output the plurality of laser beams.

In detail, for example, the laser generation device 200 may modulate at least one reference laser beam into a first laser beam which has a first energy magnitude and a first pulse duration and may then modulate the first laser beam into an nth laser beam which has an nth energy magnitude and an nth pulse duration different from those of the first laser beam. Herein, n is a natural number of 2 or more.

The pulse duration of the laser beam may be every period (or one pulse) duration when laser energy is irradiated on a periodic basis, which may be a pulse application duration before the laser beam is irradiated for each of at least one pulse and is disconnected. For example, as the pulse duration is set every 6 to 10 nanosecond (ns), the laser beam may be repeatedly output. Upon the duration modulation of the laser beam, the laser beam may be modulated and repeatedly output every 450 picosecond (p), 750 p, 3500 femtosecond (fs), or the like.

The laser generation device 200 may alternately and repeatedly output, that is, transmit a plurality of laser beams, each of which has different laser energy and a different pulse duration, among first to nth laser beams, which are autonomously generated, to the laser irradiation device 110 at intervals of a predetermined period.

Thus, the one laser irradiation device 110 may alternately and repeatedly irradiate a skin lesion with at least two types of laser beams, each of which has a different energy magnitude and a different pulse duration.

A user may select the pulse duration of the laser beam irradiated onto the laser irradiation device 110 on a stage-by-stage basis using a control button (not shown), a switch (not shown), and the like of a controller 230 of the laser generation device 200 to select and set a magnitude of irradiated energy for each stage. The user may also set a repeated irradiation period of a laser beam irradiated alternately and repeatedly for each stage.

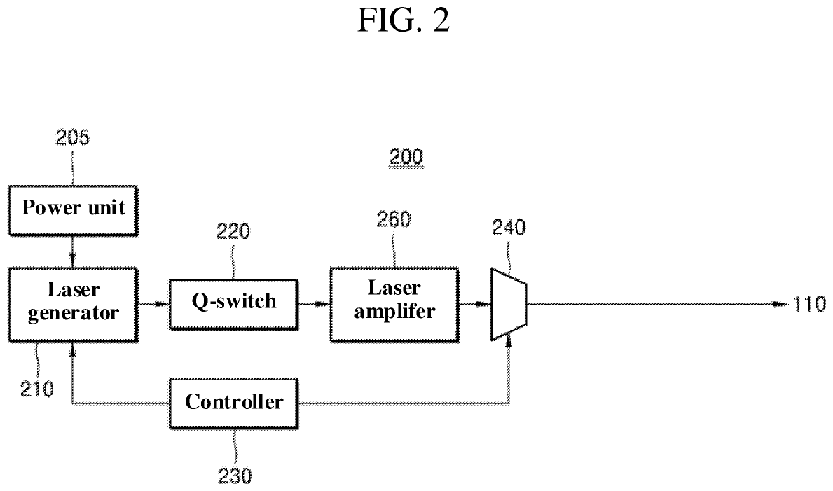

FIG. 2 is a configuration block diagram illustrating in detail a laser generation device shown in FIG. 1.

A laser generation device 200 shown in FIG. 2 may include a laser generator 210 for generating at least one reference laser beam using commercial power from a power unit 205, a Q-switch 220 for modulating and outputting the reference laser beam into first and second laser beams respectively having first and second energy magnitudes and respectively having first and second pulse durations, a laser amplifier 260 for amplifying and outputting the first and second laser beams from the Q-switch 220, and a first selective switching unit 240 for selectively transmitting the amplified first and second laser beams to a laser irradiation device 110 or an electronic switch (not shown).

The laser generator 210 may generate a reference laser beam of a wavelength range according to at least one medium using the at least one medium provided in a resonator (not shown). For example, when a neodymium-doped yttrium aluminum garnet (Nd:YAG) is provided in the resonator to generate the reference laser beam, the laser generator 210 may generate the reference laser beam of the wavelength range of 1064 nm. When modulating the wavelength range of the reference laser beam using the Nd:YAG, the laser generator 210 may generate the reference laser beam of the wavelength range of 532 nm.

The Q-switch 220 may switch (or shut) and output the reference laser beam applied from the laser generator 210 at intervals of a predetermined period (e.g., 6 to 10 nanosecond (ns)) to modulate and output the reference laser beam into the first and second laser beams respectively having different first and second energy magnitudes and respectively having different first and second pulse durations. The Q-switch 220 may be provided at a laser output end of the laser generator 210 or may be integrated with the laser generator 210.

The laser amplifier 260 may amplify and transmit the first and second laser beams, from the Q-switch 220, to the first selective switching unit 240. The laser amplifier 260 may be additionally provided if necessary.

The first selective switching unit 240 may alternately and repeatedly transmit the laser beam of the first pulse duration and the laser beam of the second pulse duration, output via the laser generator 210, to the laser irradiation device 110 under control of the controller 230. Thus, the first and second laser beams from the first selective switching unit 240 may be alternately and repeatedly input to the laser irradiation device 110. The user may repeatedly irradiate a skin lesion with the first and second laser beams, which respectively have different energy magnitudes and respectively have different pulse durations to be alternated, using the one laser irradiation device 110.

To selectively transmit the first and second laser beams to the laser irradiation device 110 alternately and repeatedly from the laser generator 210 to the first selective switching unit 240, the controller 230 may generate first and second selection signals to be alternately repeated through an adjustment plate (not shown) and may provide the first and second selection signals to the first selective switching unit 240. The number of times the first laser beam and the second laser beam are alternated and the number of times the first laser beam and the second laser beam are repeated may be reset by first and second control signals output from the controller 230. The user may select and control the number of times the first laser beam and the second laser beam are alternated and the number of times the first laser beam and the second laser beam are repeated, using a control button (not shown), a switch (not shown), and the like of the laser generation device 200.

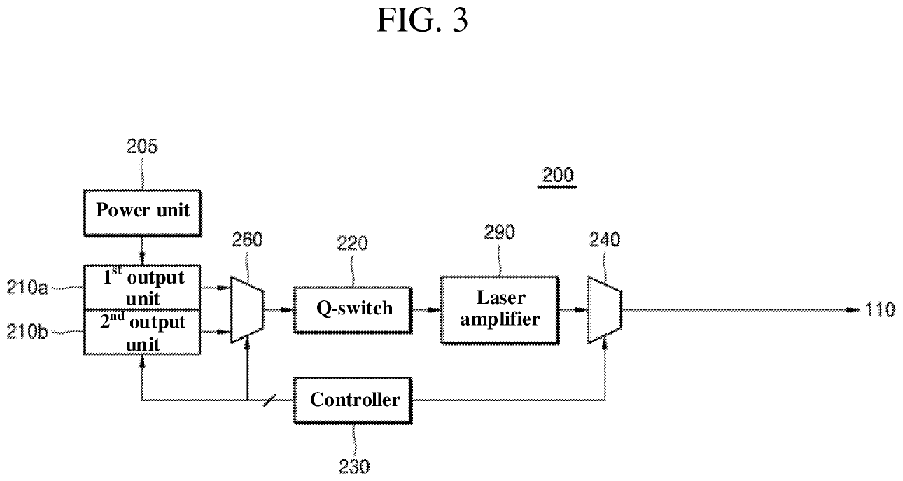

FIG. 2 is a configuration block diagram illustrating in detail a laser generation device shown in FIG. 1. FIG. 3 is another configuration block diagram illustrating in detail a laser generation device shown in FIG. 1.

Referring to FIGS. 2 and 3, a Q-switch 220 of a laser generation device 200 may switch (or shut) and output a reference laser beam (e.g., a reference laser beam of the wavelength range of 1064 nm), applied from a first output unit 210a, at intervals of 6 to 10 nanosecond (ns) to modulate and output the reference laser beam into a first laser beam which has a first energy magnitude and a first pulse duration of 6 to 10 ns. Furthermore, the Q-switch 220 may output a reference laser beam (e.g., a reference laser beam of the wavelength range of 1064 nm), applied from a second output unit 210b, at intervals of 450 picosecond (p) or 750 p to modulate and output the reference laser beam into a second laser beam which has a second energy magnitude and a second duration of 450 p or 750 p.

Meanwhile, a first selective switching unit 240 may selectively transmit the first laser beam to a laser irradiation device 110 or an electronic switch (not shown) alternately and repeatedly. In this case, when the first laser beam having the first energy magnitude and the first pulse duration of 6 to 10 ns is input to the electronic switch, the electronic switch may modulate the energy magnitude and the pulse duration of the first laser beam into the second energy magnitude and the second pulse duration of 450 p of the second laser beam different from the first laser beam and may transmit the modulated second laser beam to the laser irradiation device 110.

The controller 230 may control the generation of the first and second laser beams by the first output unit 210a for generating and outputting a first reference laser beam having the first pulse duration of 6 to 10 ns and the second output unit 210b for generating and outputting a second reference laser beam having the second pulse duration of 450 p or 750 p and may adjust the first and second laser beams to alternately and repeatedly transmit the first and second laser beams to the laser irradiation device 110 via the first selective switching unit 240.

The first selective switching unit 240 may selectively transmit the first laser beam to the laser irradiation device 110 alternately and repeatedly depending on first and second control signals from the controller 230, such that the first laser beam is alternately and repeatedly input to the laser irradiation device 110.

The number of times the first laser beam and the second laser beam are alternated and the number of times the first laser beam and the second laser beam are repeated may be reset by the first and second control signals output from the controller 230. A user may select and control the number of times the first laser beam and the second laser beam are alternated and the number of times the first laser beam and the second laser beam are repeated, using a control button (not shown), a switch (not shown), and the like of the laser generation device 200.

Referring to FIG. 3, an embodiment of the inventive concept may generate and use the first laser beam by selectively using a plurality of reference laser beams.

As described above, as an example, when an Nd:YAG is provided as a medium in a resonator (not shown) to generate a reference laser beam, a laser generator 210 may generate the first reference laser beam of the wavelength range of 1064 nm. When modulating the wavelength range of the first reference laser beam using the Nd:YAG, the laser generator 210 may generate a second reference laser beam of the wavelength range of 532 nm. Furthermore, there is a structure where it is able to perform complex treatments using various media.

To this end, the laser generator 210 according to an embodiment of the inventive concept may include the first output unit 210a for generating and outputting a first reference laser beam using a first medium such as an Nd:YAG and the second output unit 210b for generating and outputting a second reference beam having a wavelength and a pulse width different from those of the first reference laser beam using the first medium.

As the above example, the controller 230 may generate a first or second control signal for selecting the first reference laser beam of the wavelength range of 1064 nm or the second reference laser beam of the wavelength range of 532 nm and may transmit the first and second control signals to the laser generator 210 and the first selective switching unit 240. The number of times the first laser beam and the second laser beam are alternated and the number of times the first laser beam and the second laser beam are repeated may be reset by the first and second control signals output from the controller 230. The user may select and control the number of times the first laser beam and the second laser beam are alternated and the number of times the first laser beam and the second laser beam are repeated, using a control button (not shown), a switch (not shown), and the like of the laser generation device 200.

As described above, a laser amplifier 290 may be optionally and additionally provided between an output end of a Q-switch 220 and the first selective switching unit 240. The laser amplifier 290 may amplify the first laser beam output from the Q-switch 220 to a predetermined level and may output the amplified first laser beam.

The controller 230 may generate a third or fourth control signal for selecting the first reference laser beam of the wavelength range of 1064 nm or the second reference laser beam of the wavelength range of 532 nm and may transmit the third and fourth control signals to a second selective switching unit 260.

Thus, the second selective switching unit 260 may transmit the first reference laser beam of the wavelength range of 1064 nm or the second reference laser beam of the wavelength range of 532 nm to the Q-switch 220 in response to the third or fourth control signal.

The Q-switch 220 may modulate the first reference laser beam or the second reference laser beam, which is selectively input from the second selective switching unit 260, into a first laser beam having a first energy magnitude and a first pulse duration and may transmit the first laser beam to the first selective switching unit 240.

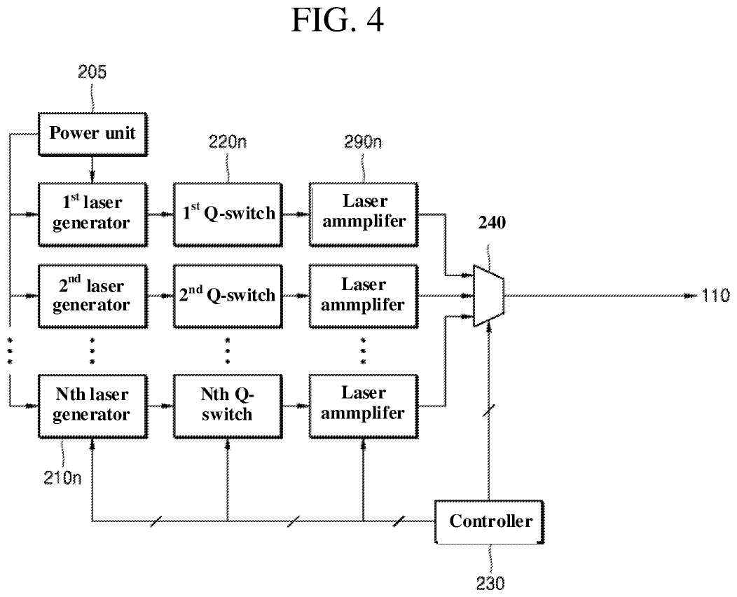

FIG. 4 is another configuration block diagram illustrating in detail a laser generation device shown in FIG. 1.

Referring to FIG. 4, an embodiment of the inventive concept may generate a plurality of reference laser beams using a plurality of different media and may generate and use a first laser beam by selectively using the plurality of reference laser beams.

For example, when an Nd:YAG is provided as a medium in a resonator (not shown) to generate a first reference laser beam, a first laser generator may generate the first reference laser beam of the wavelength range of 1064 nm.

For another example, when Alexandrite is provided as a medium in another resonator (not shown) to generate a second reference laser beam, a second laser generator may generate the second reference laser beam of the wavelength range of 755 nm.

For another example, when copper bromide is provided as a medium in another resonator (not shown) to generate a third reference laser beam, a third laser generator may generate the third reference laser beam of the wavelength range of 578 nm.

A laser generator 210 of FIG. 2 may generate a plurality of reference laser beams using a plurality of different media in such a manner to selectively use the plurality of reference laser beams. To this end, as shown in FIG. 4, the laser generator 210 may include a first laser generator for generating and outputting a first reference laser beam using a first medium and an nth laser generator 210n for generating and outputting an nth reference laser beam using a medium different from the first medium.

The first to nth laser generators 210n may transmit first to nth reference laser beams to first to nth Q-switches 220n, respectively. The first to nth Q-switches 220n may transmit the first to nth reference laser beams input thereto to a first selective switching unit 240. In this case, the first to nth Q-switches 220n may amplify levels of the first to nth reference laser beams using at least one laser amplifier 290n and may transmit the amplified first to nth reference laser beams to the first selective switching unit 240.

The first selective switching unit 240 may selectively output the first to nth reference laser beams to a laser irradiation device 110 depending on a selection of the controller 230.

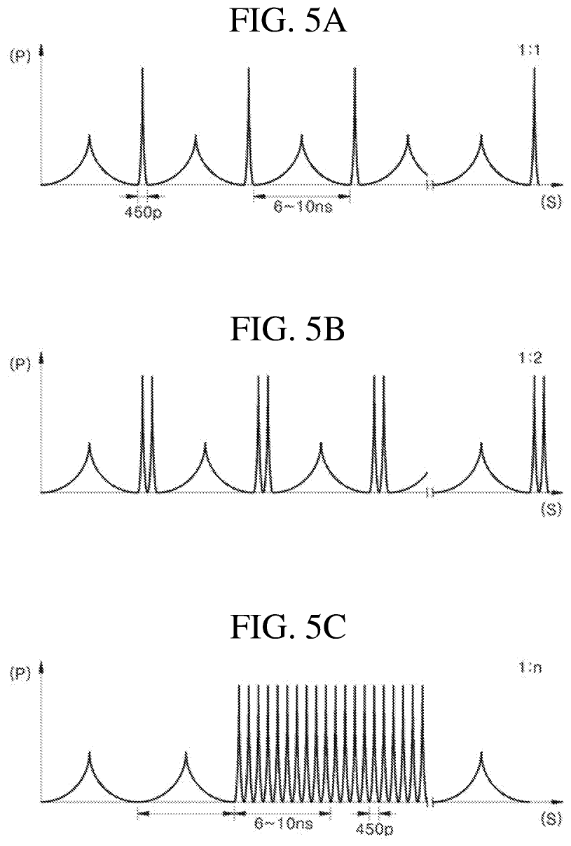

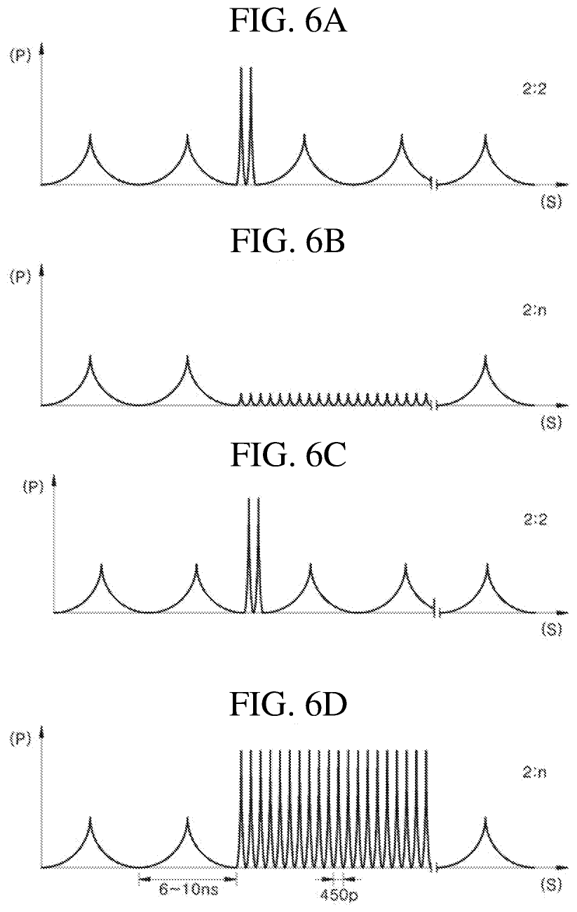

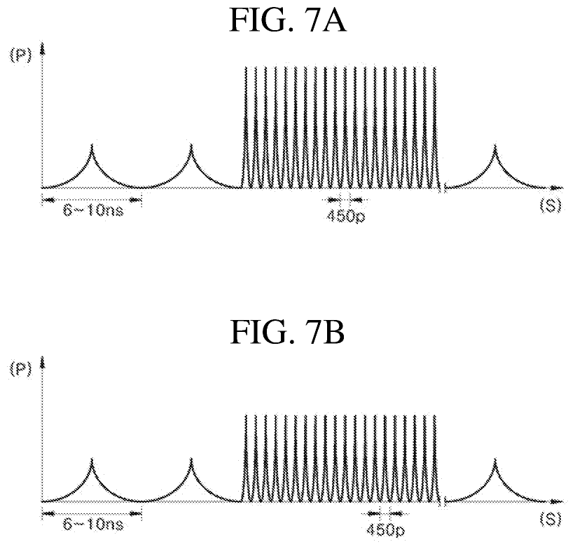

FIGS. 5A to 5C are drawings illustrating changes in pulse duration of a laser beam applied from a laser generation device shown in FIGS. 1 and 2 to a laser irradiation device. FIGS. 6A to 6D are drawings illustrating changes in pulse duration of a laser beam applied from a laser generation device shown in FIGS. 1 and 3 to a laser irradiation device. FIGS. 7A and 7B are drawings illustrating changes in pulse duration of a laser beam applied from a laser generation device shown in FIGS. 1 and 4 to a laser irradiation device.

As shown in FIGS. 5A to 5C, an embodiment of the inventive concept is exemplified as a first pulse duration of a first laser beam is set to 6 to 10 ns and a second pulse duration of a second laser beam is set to 450 p.

A first selective switching unit 240 of FIG. 2 may selectively transmit a first laser beam to a laser irradiation device 110 or an electronic switch alternately or repeatedly depending on first and second control signals from a controller 230 of FIG. 2, as shown in FIGS. 5A to 5C, such that the first laser beam from the first selective switching unit 240 and a second laser beam modulated through the electronic switch are alternately and repeatedly input to the laser irradiation device 110.

The number of times the first laser beam and the second laser beam are alternated and the number of times the first laser beam and the second laser beam are repeated may be reset by the first and second control signals output from the controller 230. A user may select and control the number of times the first laser beam and the second laser beam are alternated and the number of times the first laser beam and the second laser beam are repeated, using a control button, a switch, and the like of a laser generation device 200 of FIG. 1. In other words, as shown in FIG. 5A, the first laser beam and the second laser beam may be selected and set to be alternated in the ratio of 1:1 and be repeatedly output. On the other hand, as shown in FIG. 5B, the first laser beam and the second laser beam may be selected and set to be alternated in the ratio of 1:1 and be repeatedly output. As shown in FIG. 5C, the first laser beam and the second laser beam may be selected and set to be alternated in the ratio of 1:n and be repeatedly output.

As shown in FIGS. 6A to 6D, when a first reference laser beam of the wavelength range of 1064 nm is applied to a Q-switch 220 of FIG. 3 by a second selective switching unit 260 of FIG. 3, the Q-switch 220 may switch (or shut) and output the first reference laser beam of the wavelength range of 1064 nm at intervals of 6 to 10 nanosecond (ns) to modulate and output the first reference laser beam into a first laser beam having a first energy magnitude p and a first pulse duration of 6 to 10 ns. In this case, as shown in FIG. 6A, the first laser beam and the second laser beam may be selected and set to be alternated in the ratio of 2:2 and be repeatedly output. In addition, as shown in FIG. 6B, the first laser beam and the second laser beam may be selected and set to be alternated in the ratio of 2:n and be repeatedly output.

The first laser beam and the second laser beam, which are generated using the first reference laser beam of the wavelength range of 1064 nm, may be alternately and repeatedly transmitted to a laser irradiation device 110 of FIG. 1.

Meanwhile, as shown in FIG. 6C, when a second reference laser beam of the wavelength range of 532 nm is applied to the Q-switch 220 by the second selective switching unit 260, the Q-switch 220 may switch (shut) and output the second reference laser beam of the wavelength range of 532 nm at intervals of 6 to 10 nanosecond (ns) to modulate and output the second reference laser beam into a first laser beam having a first energy magnitude p and a first pulse duration of 6 to 10 ns.

On the other hand, the electronic switch may modulate the energy magnitude p and the pulse duration s of the first laser beam having the first energy magnitude p and the first pulse duration of 6 to 10 ns into a second energy magnitude and a second pulse duration of 450 p of a second laser beam different from the first laser beam and may transmit the modulated second laser beam to the laser irradiation device 110. Thus, the first laser beam and the second laser beam, which are generated using the second reference laser beam of the wavelength range of 532 nm, may be alternately and repeatedly transmitted to the laser irradiation device 110. In this case, as shown in FIG. 6C, the first laser beam and the second laser beam may be selected and set to be alternated in the ratio of 2:2 and be repeatedly output. In addition, as shown in FIG. 6D, the first laser beam and the second laser beam may be selected and set to be alternated in the ratio of 2:n and be repeatedly output.

As shown in FIG. 7A, when a first reference laser beam of the wavelength range of 1064 nm is applied to the Q-switch 220 by the second selective switching unit 260, the Q-switch 220 may switch (or shut) the first reference laser beam of the wavelength range of 1064 nm at intervals of 6 to 10 nanosecond (ns) to modulate and output the first reference laser beam into a first laser beam having a first energy magnitude p and a first pulse duration of 6 to 10 ns.

On the other hand, the electronic switch may modulate the energy magnitude p and the pulse duration s of the first laser beam having the first energy magnitude p and the first pulse duration of 6 to 10 ns into a second energy magnitude and a second pulse duration of 450 p of a second laser beam different from the first laser beam and may transmit the modulated second laser beam to the laser irradiation device 110.

Thus, the first laser beam and the second laser beam, which are generated using the first reference laser beam of the wavelength range of 1064 nm, may be alternately and repeatedly transmitted to the laser irradiation device 110.

Meanwhile, as shown in FIG. 7B, when a second reference laser beam of the wavelength range of 755 nm is applied to the Q-switch 220 by the second selective switching unit 260, the Q-switch 220 may switch (shut) and output the second reference laser beam at intervals of 6 to 10 nanosecond (ns) to modulate and output the second reference laser beam into a first laser beam having a first energy magnitude p and a first pulse duration of 6 to 10 ns.

On the other hand, the electronic switch may modulate the energy magnitude p and the pulse duration s of the first laser beam having the first energy magnitude p and the first pulse duration of 6 to 10 ns into a second energy magnitude and a second pulse duration of 450 p of a second laser beam different from the first laser beam and may transmit the modulated second laser beam to the laser irradiation device 110.

Thus, the first laser beam and the second laser beam, which are generated using the second reference laser beam of the wavelength range of 755 nm, may be alternately and repeatedly transmitted to the laser irradiation device 110.

As such, the skin treatment laser apparatus 101 capable of performing pulse duration modulation according to an embodiment of the inventive concept having the above-mentioned technical features may alternately and repeatedly radiate laser beams of different pulse durations during a laser irradiation period according to a chromophore.

Thus, laser beams of different pulse durations may be alternately and repeatedly irradiated using the one laser irradiation device 110, thus actually shortening a laser treatment time and improving the efficiency of treatment.

The skin treatment laser apparatus using the complex irradiations with the different pulse durations according to an embodiment of the inventive concept having the above various technical features may alternately and repeatedly radiate laser beams of different pulse durations during a laser irradiation period according to a chromophore.

Thus, the skin treatment laser apparatus may alternately and repeatedly radiate laser beams of different pulse durations using one laser irradiation device, thus actually shortening a laser treatment time and improving the efficiency of treatment.

It should be understood that the examples stated above are illustrative in every way, not limitative. The scope of the inventive concept is defined by the following claims, and all modified or varied forms derived from the meaning and scope of the claims and also equivalent concepts thereof should be interpreted to be included in the scope of the present invention.

What is claimed is:

1. A skin treatment laser apparatus using complex irradiations with different pulse durations, the apparatus comprising:
a laser generation device configured to modulate at least one reference laser beam into a plurality of laser beams, each of which has a different energy magnitude and a different pulse duration, and alternately and repeatedly output the plurality of laser beams; and
a laser irradiation device configured to irradiate a skin lesion with the plurality of laser beams alternately and repeatedly applied from the laser generation device.

2. The apparatus of claim 1, wherein the laser generation device modulates the at least one reference laser beam into a first laser beam having a first energy magnitude and a first pulse duration and an nth laser beam having an nth energy magnitude and an nth pulse duration different from those of the first laser beam and alternately and repeatedly outputs the first to nth laser beams at intervals of a predetermined period.

3. The apparatus of claim 1, wherein the laser generation device includes:
a laser generator configured to generate the at least one reference laser beam using commercial power from a power unit;
a Q-switch configured to modulate and output the reference laser beam from the laser generator into first and second laser beams respectively having first and second energy magnitudes and respectively having first and second pulse durations; and
a first selective switching unit configured to selectively transmit the first and second laser beams to the laser irradiation device alternately and repeatedly, depending on a selection of the controller, such that the first and second laser beams are alternately and repeatedly transmitted to the laser irradiation device.

4. The apparatus of claim 3, wherein the controller generates and provides first and second selection signals to the first selective switching unit, such that the first and second laser beams are alternately repeated at intervals of a predetermined period, and
wherein the first selective switching unit selectively transmits the first or second laser beam to the laser irradiation device in response to the first and second selection signals alternately and repeatedly input from the controller.

5. The apparatus of claim 3, wherein the laser generator includes:
a first output unit configured to generate and output a first reference laser beam using a first medium; and
a second output unit configured to generate and output a second reference laser beam having a wavelength and a pulse width different from those of the first reference laser beam, using the first medium.

6. The apparatus of claim 5, wherein the Q-switch modulates the first and second reference laser beams from the first output unit and the second output unit into the first and second laser beams respectively having the first and second energy magnitudes and respectively having the first and second pulse durations and transmits the first and second laser beams to the first selective switching unit.

7. The apparatus of claim 3, wherein the laser generator includes:
a first laser generator configured to generate and output a first reference laser beam using a first medium; and
an nth laser generator configured to generate and output an nth reference laser beam using a medium different from the first medium, and
wherein the first selective switching unit selectively transmits the first to nth reference laser beams to the laser irradiation device or an electronic switch alternately and repeatedly, depending on a selection of the controller, such that the first to nth laser beams are alternately and repeatedly transmitted to the laser irradiation device.
